(19) United States
(12) Patent Application Publication
(10) Pub. No.: US 2022/0270741 A1
(43) Pub. Date: Aug. 25, 2022

(54) HEALTH MANAGEMENT SUPPORT APPARATUS, METHOD, AND PROGRAM

(71) Applicant: NIPPON TELEGRAPH AND TELEPHONE CORPORATION, Tokyo (JP)

(72) Inventors: Reiko ARUGA, Musashino-shi, Tokyo (JP); Akihiro CHIBA, Musashino-shi, Tokyo (JP); Naoki ASANOMA, Musashino-shi, Tokyo (JP); Shigekuni KONDO, Musashino-shi, Tokyo (JP)

(73) Assignee: NIPPON TELEGRAPH AND TELEPHONE CORPORATION, Tokyo (JP)

(21) Appl. No.: 17/632,015

(22) PCT Filed: Aug. 5, 2019
(86) PCT No.: PCT/JP2019/030744

§ 371 Date: Feb. 1, 2022

(51) Int. Cl. G16H 20/60

(52) U.S. Cl. CPC G16H 20/60

(57) ABSTRACT

According to one embodiment of the present invention, food item information on a plurality of food items suitable for snacking classified into a plurality of clusters according to similarity in feature quantities for the shape, taste, and texture is stored. Calories less than the difference between the sum of the calorie consumption by the user during a first period before user-specified desired snacking time and expected calorie consumption during a second period thereafter and the sum of calorie intake during the first period and expected calorie intake during the second period by the user are obtained as ingestible snacking calories, and a food item having calories less than the ingestible snacking calories and belonging to a cluster other than a cluster selected for the previous snacking is selected from the food item information and presented to the user.

### TECHNICAL FIELD

An embodiment of the present invention relates to a health care support device, method, and program for example for supporting human health care efforts.

### BACKGROUND ART

In recent years, the number of people suspected of having metabolic syndrome or lifestyle-related diseases has been increasing, and awareness of health care has been growing. Meanwhile, various attempts to support human health care have been proposed. One of known examples is a method to maintain a healthy lifestyle by selecting appropriate food items for example according to calorie consumption and the amount of exercise. According to a method disclosed in NPL 1 as one such example, an evaluation value is calculated for daily calorie intake in consideration of events included in the user's schedule, and recipe candidates for successful dieting within a target period are selected and recommended on the basis of the evaluation value and a database of meal recipes.

### CITATION LIST

### Non Patent Literature

[NPL 1] Yoko Mino, Ichiro Kobayashi, “Flexible Recommendation of Cooking Recipes for a Diet”, Journal of Japan Society for Fuzzy Theory and Intelligent Informatics, Vol. 24, No. 1, pp. 616-626, 2012

### SUMMARY OF THE INVENTION

### Technical Problem

According to the method disclosed in NPL 1, however, when the evaluation value for the user's daily calory intake is the same for a number of days, the same or similar recipes may be recommended for these days. In this case, the user may be tired of the recommended recipes, and the efforts for health care may not last long.

With the foregoing in view, it is an object of the present invention to provide a technique for effectively supporting the user's health care efforts by recommending food items of which the user is unlikely to be tired.

### Means for Solving the Problem

According to one aspect of the invention, a health care support device includes a memory and a hardware processer, the memory includes a storage unit which stores information representing a plurality of food items in association with calories contained therein as the plurality of food items is classified into a plurality of clusters according to similarity in a feature quantity thereof. The hardware processor is configured to carry out calculating ingestible calories which can be ingested at user-specified desired intake time according to the difference between estimated calorie consumption and estimated calorie intake by the user during a previously set period, selecting, from the storage unit, a food item having contained calories less than the calculated ingestible calories and belonging to a cluster other than a cluster from which a food item has been selected at time before the desired intake time, and outputting the information representing the selected food item.

### Effects of the Invention

According to another aspect of the invention, a plurality of food items are classified into a plurality of clusters according to similarity in feature quantities thereof, and when a food item which can be ingested at user-specified desired intake time for a snack is selected, a food item belonging to a cluster other than the previously selected cluster is selected. Therefore, a food item of which the user is less likely to be tired can be selected, which can effectively support the user's health care efforts.

### BRIEF DESCRIPTION OF DRAWINGS

FIG. 1 is a block diagram of the hardware structure of a mobile information terminal used as a health care support device according to one embodiment of the present invention.

FIG. 2 is a block diagram of the software configuration of the mobile information terminal used as the health care support device according to the embodiment of the present invention.

FIG. 3 is an exemplary view of food item information stored in a food item information storage unit provided in the storage unit of the mobile information terminal shown in FIGS. 1 and 2.

FIG. 4 is a view for illustrating the definition of a shape ID included in the food item information shown in FIG. 3.

FIG. 5 is a view for illustrating the definition of a taste ID included in the food item information shown in FIG. 3.

FIG. 6 is a view for illustrating the definition of a texture ID included in the food item information shown in FIG. 3.

FIG. 7 is a flowchart for illustrating the processing procedure and processing content in food item recommendation control carried out by the mobile information terminal shown in FIG. 2.

FIG. 8 is a flowchart for illustrating the processing procedure and processing content of ingestible snacking calorie calculation in the processing procedure shown in FIG. 7.

FIG. 9 is a flowchart for illustrating the processing procedure and processing content of recommended food item information selection in the processing procedure shown in FIG. 7.

FIG. 10 shows exemplary function data for use in obtaining a coefficient α in the ingestible snacking calorie calculation shown in FIG. 8.

FIG. 11 shows exemplary function data for use in obtaining a coefficient β in the ingestible snacking calorie calculation shown in FIG. 8.

FIG. 12 illustrates display data in a first step displayed by display processing in the processing procedure shown in FIG. 7.

FIG. 13 illustrates display data in a second step displayed by display processing in the processing procedure shown in FIG. 7.

FIG. 14 illustrates display data in a third step displayed by the display processing in the processing procedure shown in FIG. 7.

FIG. 15 illustrates display data in a fourth step displayed by the display processing in the processing procedure shown in FIG. 7.

### DESCRIPTION OF EMBODIMENTS

Hereinafter, an embodiment of the present invention will be described in conjunction with the drawings.

### Embodiment

### (Exemplary Configuration)

### (1) Mobile Information Terminal UT

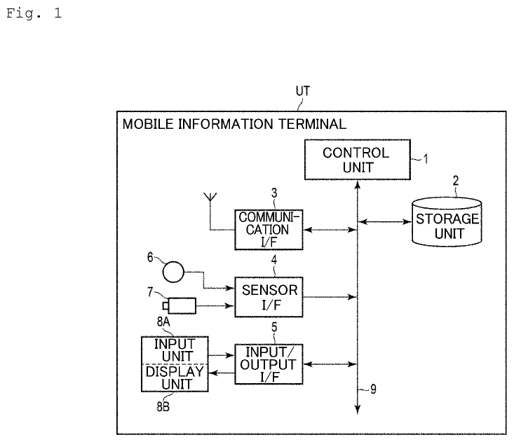

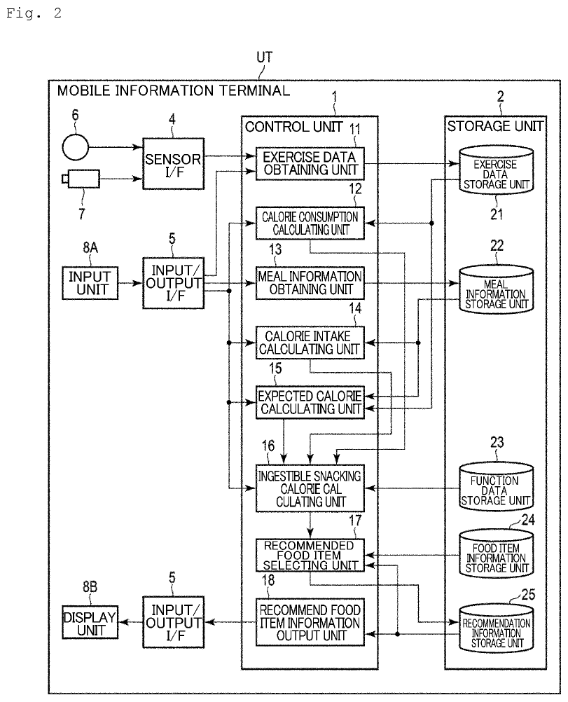

FIGS. 1 and 2 are block diagrams of the hardware and software configurations of a mobile information terminal UT used as a health care support device according to one embodiment of the present invention.

The mobile information terminal UT may be a smartphone, a tablet type terminal, or a note type personal computer. The mobile information terminal UT includes a control unit 1 having a hardware processor such as a CPU (Central Processing Unit), and the control unit 1 is connected with a storage unit 2, a communication interface (communication I/F) 3, a sensor interface (sensor I/F) 4, and an input/output interface (input/output I/F) 5 through a bus 9.

The communication I/F 3 may include an interface compatible with a wireless LAN, a short-range wireless data communication standard (such as Bluetooth (registered trademark)) or a public mobile communication network and transmits data to/from a website for example via a network which is not shown. The communication I/F 3 may also include an interface compatible with a wired network such as a public wired network, a wired LAN, or a CATV network.

The sensor I/F 4 is connected with a motion sensor 6. The motion sensor 6 may include an acceleration sensor or an angular rate sensor (gyro sensor) and is used to detect the motion of the mobile information terminal UT. Specifically, the sensor detects the motion of the mobile information terminal UT in the six axis directions. The sensor I/F 4 converts a detection signal from the motion sensor 6 into digital data and outputs the digital data to the control unit 1 as data representing the walking exercise of the user together with a time stamp indicating the detection time.

A camera 7 is also connected to the sensor I/F 4. The camera 7 is used for example to capture images of meal menus and recipes. The sensor I/F 4 outputs image data on a menu or a recipe for a meal output from the camera to the control unit 1 as meal information together with a time stamp indicating the time of capture.

An input unit 8A and a display unit 8B are connected to the input/output I/F 5. The input unit 8A and the display unit 8B may include a so-called tablet-type device including an LCD (Liquid Cristal Display) or an organic EL (Electro Luminescence) display screen and an input sheet provided thereon. Any other user interface device such as various switches, a microphone, and a speaker may be connected to the input/output I/F 5.

The storage unit 2 may include, as a storage medium, a combination of a non-volatile memory which allows random writing and reading such as an HDD (Hard Disk Drive) and an SSD (Solid State Drive), a non-volatile memory such as a ROM (Read Only Memory), and a volatile memory such as a RAM (Random Access Memory). The storage area of the storage unit includes a program storage area and a data storage area. The program storage area stores middleware such as an operating system (OS) as well as application programs necessary for executing various kinds of control processing according to the embodiment of the present invention.

The data storage area includes an exercise data storage unit 21, a meal information storage unit 22, a function data storage unit 23, a food item information storage unit 24, and a recommendation information storage unit 25.

The exercise data storage unit 21 is used to store data representing walking exercise output from the sensor I/F 4.

The meal information storage unit 22 is used to store meal information received by the control unit 1 from the input/output I/F 5 or the sensor I/F 4.

The function data storage unit 23 is used to store function data for obtaining coefficients α and β to be used in calculating ingestible snacking calories which will be described.

The recommendation information storage unit 25 stores recommended food item information selected by the control unit 1 (which will be described) as suitable food items for snacking. The recommendation information storage unit 25 also stores information representing the history of the food item information selection in the past.

The food item information storage unit 24 stores attribute information about a plurality of food items suitable for snacking. FIG. 3 shows exemplary attribute information. The attribute information for each food item for example includes a “food item name”, “calories” contained therein, the “initial letter” of the food item name, a “shape ID”, a “taste ID”, a “texture ID”, a “cluster ID”, and the “image ID” of the food item in association with food item ID.

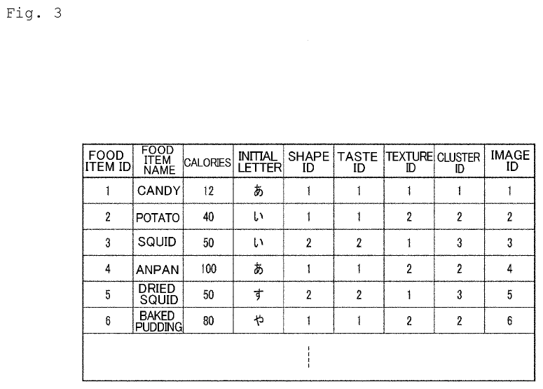

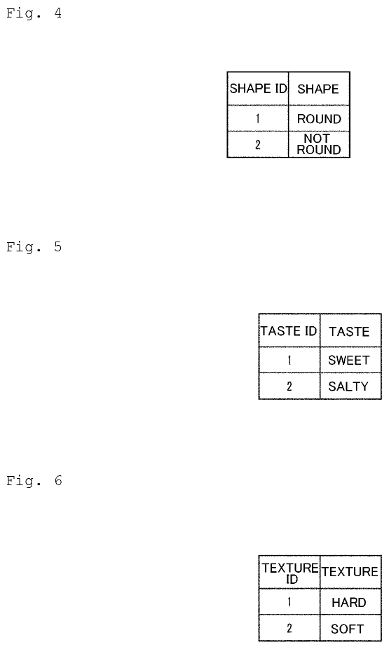

For example as shown in FIG. 4, the “shape ID” defines one of two kinds of product shapes, “round” and “not round”, and different shape IDs (for example, shape ID=1, 2) are assigned to these shapes. For example, as shown in FIG. 5, the “taste ID” defines one of two kinds of tastes, “sweet” and “salty”, and taste IDs (for example, taste ID=1 or 2) are assigned to these tastes. For example, as shown in FIG. 6, the “texture ID” defines two kinds of texture, “hard” and “soft”, and texture IDs (for example texture ID=1, 2) are assigned to these textures.

The food items are classified into multiple clusters according to similarity in three feature quantities for the “shape”, the “taste”, and the “texture”. The “cluster IDs” identify these clusters. In this example, the total score of a “shape ID”, a “taste ID”, and a “texture ID” as feature quantities is obtained, and food items with the same total score are classified as belonging to the same cluster.

The food item information can be obtained for example by downloading, through the communication I/F 3, food item information prepared in advance and uploaded on a website by a food manufacturer or a service provider. The food item information may also be additionally stored in the food item information storage unit 24 as the user manually inputs information about the user's desired food item using for example the input unit 8A and the camera 7.

The control unit 1 has, as control functions for carrying out the embodiment of the invention, an exercise data obtaining unit 11, a calorie consumption calculating unit 12, a meal information obtaining unit 13, a calorie intake calculating unit 14, an expected calorie calculating unit 15, an ingestible snacking calorie calculating unit 16, a recommended food item selecting unit 17, and a recommended food item information output unit 18. All these control functions are implemented by causing the hardware processor of the control unit 1 to execute an application program stored in the program storage area in the storage unit 2.

The exercise data obtaining unit 11 obtains exercise data output from the sensor I/F 4 during the walking period of the user together with a time stamp indicating the time of detection and stores the exercise data in the exercise data storage unit 21 together with the time stamp as data representing the user's walking exercise. When the user manually inputs the number of steps and a time stamp indicating the walking period using the input unit 8A, the exercise data obtaining unit 11 also obtains the input number of steps and the time stamp from the input/output I/F 5 and has them stored in the exercise data storage unit 21.

When the user specifies desired intake time for a snack (also referred to as desired snacking time), the calorie consumption calculating unit 12 calculates calorie consumption by the user during the period from for example the time of rising to the desired intake time (a first period) on the basis of the exercise data stored in the exercise data storage unit 21. A conversion formula which converts the number of steps into calorie consumption is used to calculate the calorie consumption. A specific example the formula will be described later.

When the user inputs meal information such as a menu or a recipe for a meal together with a time stamp representing the mealtime using the input unit 8A, the meal information obtaining unit 13 obtains the meal information and the time stamp from the input/output I/F 5 and has them stored in the meal information storage unit 22. When the user captures an image representing a menu or a recipe for a meal using the camera 7, the meal information obtaining unit 13 obtains the image data together with a time stamp representing the time of capture from the sensor I/F 4 and has them stored in the meal information storage unit 22.

The calorie intake calculating unit 14 calculates calorie intake by the user during the period from the time of rising to the desired intake time for a snack (the first period) according to meal information stored in the meal information storage unit 22.

The expected calorie calculating unit 15 calculates calories expected to be consumed during the day (the second period) from the desired intake time for a snack and expected calorie intake during the second period. The expected calorie consumption and expected calorie intake can be calculated for example from exercise data during the second period up to the day before stored in exercise data storage unit 21 and meal information during the second period up to the day before stored in the meal information storage unit 22. When the user inputs an expected number of steps and scheduled meal information during the second period by the input unit 8A, the expected calorie calculating unit 15 calculates the expected calorie consumption and the expected calorie intake according to the input expected number of steps and the scheduled meal information.

The ingestible snacking calorie calculating unit 16 calculates ingestible calories at the user specified desired intake time for a snack according to the calorie consumption during the first period calculated by the calorie consumption calculating unit 12, the calorie intake during the first period calculated by the calorie intake calculating unit 14, and the expected calorie consumption and the expected calorie intake during the second period calculated by the expected calorie calculating unit 15, and function data stored in the function data storage unit 23. Note that a specific example of the calculation of the ingestible calories will be described in the following.

The recommended food item selecting unit 17 refers to food item information stored in the food item information storage unit 24 and the history of food item information selection in the past stored in the recommendation information storage unit 25 and selects a food item which satisfies the condition of the ingestible calories calculated by the ingestible snacking calorie calculating unit 16. Then, the selected recommended food item information is stored in the recommendation information storage unit 25.

The recommended food item information output unit 18 displays the recommended food item information stored in the recommendation information storage unit 25 on the display unit 8B according to prescribed display rules.

(Exemplary Operation)

Now, the operation of recommending a food item suitable for snacking by the mobile information terminal UT configured as described above will be described.

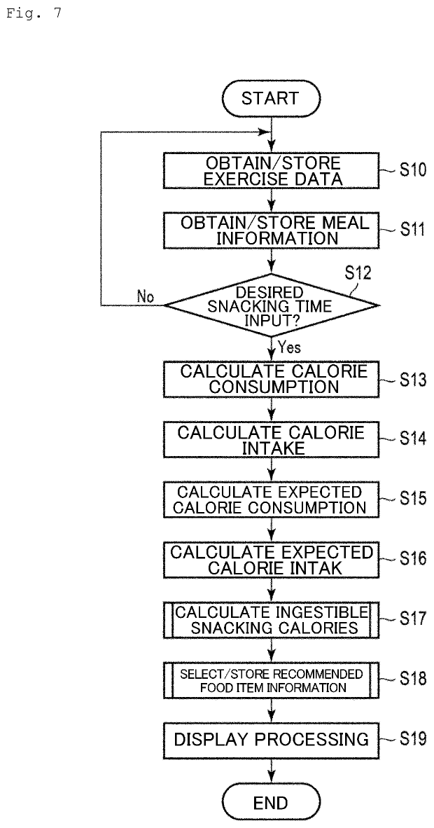

FIG. 7 is a flowchart for illustrating the processing procedure and processing content of food item recommendation control executed by the mobile information terminal UT. In the following description, it is assumed that attribute information about a plurality of food items suitable for snacking is stored in the food item information storage unit 24 as the items are classified into a plurality of clusters (cluster ID=1, 2, 3) as illustrated in FIG. 3.

(1) Obtaining Exercise Data and Meal Information

Before starting to walk, the user first inputs his/her own weight and stride using the input unit 8A as information necessary for converting the number of steps into calorie consumption and stores the information in the exercise data storage unit 21.

When the user does walking exercise while carrying the mobile information terminal UT, the motion is detected by the sensor 6, and the detection signal is converted into digital data as motion data by the sensor I/F 4. The control unit 1, under the control of the exercise data obtaining unit 11, obtains the motion data from the sensor I/F 4 in step S10, determines the period during which the user keeps walking on the basis of the motion data and has the motion data obtained during the period stored in the exercise data storage unit 21 as data representing the user's walking exercise together with a time stamp representing the detection time.

Meanwhile, when the user inputs a step number value measured by for example a pedometer separately carried by the user and a time stamp indicating the walking period by the input unit 8A, the control unit 1, under the control of the exercise data obtaining unit 11, also takes in the input step number value and the time stamp for the walking period from the input/output I/F 5 and has them stored in the exercise data storage unit 21.

The exercise data may be obtained only from the detection data by the sensor 6 or may be obtained only from input operation by the user.

Assume that the user inputs meal information for example indicating a menu or a recipe for a meal after the meal together with a time stamp representing the mealtime using the input unit 8A. In this case, the control unit 1, under the control of the meal information obtaining unit 13, takes in the input meal information and the time stamp from the input/output I/F 5 in step S11 and has them stored in the meal information storage unit 22.

Meanwhile, when the user captures an image indicating a menu or a recipe for a meal using the camera 7, the control unit 1, under the control of the meal information obtaining unit 13, takes in the image data together with a time stamp representing the time of capture from the sensor I/F 4 and has them stored in the meal information storage unit 22.

The meal information may be obtained only from the input unit 8A or only from the camera.

The control unit 1 repeatedly obtains the exercise data and the meal information.

(2) Calculation of Calorie Consumption and Calorie Intake

Assume that the user tries to have a snack and inputs for example desired intake time for having a between-meal-snack (desired snacking time) “15:00” using the input unit 8A. Upon receiving input of the desired snacking time in step S12, the control unit 1 calculates in step S13, under the control of the calorie consumption calculating unit 12, calorie consumption by the user during the period from the time of rising to the desired snacking time (the first period) on the basis of exercise data stored in the exercise data storage unit 21.

The calorie consumption is calculated, for example, using the following conversion formula:

Calorie consumption [kcal]=METs×body weight [kg]×number of steps [steps]×(stride [m/step]÷1000)÷speed [km/hour]×1.05

Note that the METs can be calculated according to the METs table. The METs table shows the relation between METs and speed for each type of exercise and is available from the website (http://www.nibiohn.go.jp/files/2011mets.pdf). The speed can be calculated from the number of steps, the stride, and the walking time or from the moving distance and the walking time if the moving distance can be detected by a GPS (Global Positioning System) sensor. The conversion formula for calculating the calorie consumption from the number of steps is not limited to the above formula and any other conversion formula may be used.

The control unit 1 also calculates in step S14, under the control of the calorie intake calculating unit 14, calorie intake by the user during the first period on the basis of meal information stored in the meal information storage unit 22. The calorie intake can be calculated, for example, from the unit calorie of each food ingredient included in the meal information and the quantity of the food ingredient. The unit calories of each food ingredient are available from a website, for example. The amount of each ingredient can be estimated for example from the number and weight of each food ingredient described in the recipe. The size of each food ingredient can be detected for example from image data on the meal by image recognition, and the amount of the food ingredient can be estimated on the basis of the size.

(3) Calculation of Expected Calorie Consumption and Expected Calorie Intake

Then, the control unit 1 first calculates in step S15, under the control of the expected calorie calculating unit 15, expected calorie consumption expected to be consumed by the user for example during the period from the desired snacking time to the bedtime of the day (the second period). For example, the average value of exercise data obtained during the second period for each day up to the day before is calculated on the basis of the exercise data for each day up to the day before stored in the exercise data storage unit 21, and then the expected calorie consumption is calculated on the basis of the average value of the exercise data assuming that the same level of walking exercise as that in the same period up to the day before will be done on the day.

When the user inputs a value for the number of steps expected during the second period on the day using the input unit 8A, the expected calorie consumption may be calculated on the basis of the input expected value for the number of steps. As the expected calorie consumption, a fixed value set in advance may be stored in the storage unit 2 and used.

The control unit 1 also calculates in step S16, under the control of the expected calorie calculating unit 15, the expected calorie intake expected to be taken by the user for example during the period from the desired snacking time to the bedtime of the day (the second period). For example, on the basis of meal information up to the day before stored in the meal information storage unit 22, the tendency of the meals taken by the user during the second period is determined for each day up to the day before, and assuming that meals with similar tendencies to those taken up to the day before will be consumed on the day, the expected calorie intake is calculated on the basis of the tendencies of the meals.

When the user inputs information representing a menu or a recipe for the meal which the user intends to take during the second period on the day using the input unit 8A, the expected calorie intake may be calculated on the basis of the input meal information. In addition, a previously set fixed value may be stored in the storage unit 1 as the expected calorie intake, and the fixed value may be used.

(4) Calculation of Ingestible Snacking Calories

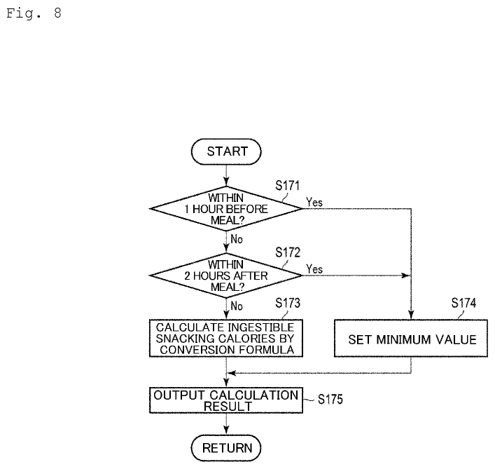

The control unit 1 calculates ingestible snacking calories as follows in step S17 under the control of the ingestible snacking calorie calculating unit 16. FIG. 8 is a flowchart for illustrating the processing procedure and processing content.

More specifically, in step S171, the ingestible snacking calorie calculating unit 16 first determines whether desired snacking time specified by the user is within one hour before the scheduled time of the nearest meal (one of the three meals of the day). When the desired snacking time is within one hour before the scheduled time of the nearest meal as a result of the determination, the timing of snacking will be just before the meal, and the process proceeds to step S174 to set a predefined minimum value as the ingestible calories.

On the other hand, when the desired snacking time is more than one hour before the nearest scheduled mealtime, the ingestible snacking calorie calculating unit 16 determines in step S172 whether the desired snacking time is within two hours after the end of the most recent meal. When the desired snacking time is within two hours after the end of the most recent meal as the result of determination, the timing of snacking will be immediately after the meal, and the process proceeds to step S174 to set the minimum value as the ingestible calories also in this case.

Meanwhile, when the desired snacking time is neither immediately before nor immediately after the nearest meal, the ingestible snacking calorie calculating unit 16 calculates ingestible calories according to a conversion formula stored in advance in the storage unit 2 in step S173. The conversion formula is for example as follows:

Ingestible snacking calories [kcal]=α×(calorie consumption [kcal]+β×expected calorie consumption [kcal])−(calorie intake [kcal]+expected calorie intake [kcal])

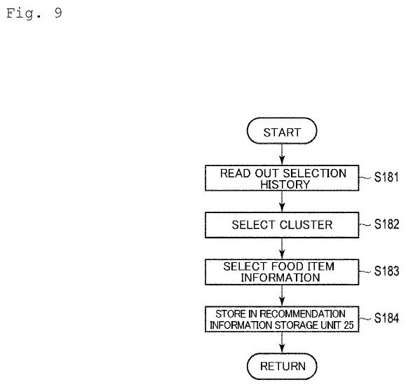

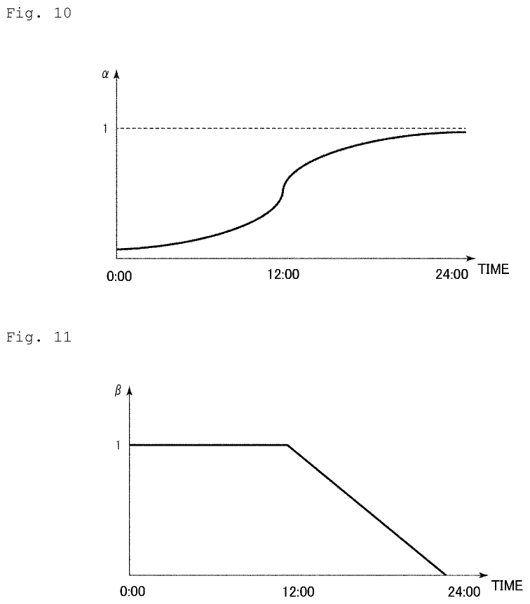

Here, the coefficients α and β are each expressed as a function of time, and the function data is stored in the function data storage unit 23. FIGS. 10 and 11 show examples thereof in which α increases with the elapse of time within the day, and β decreases with the elapse of time within the day. The ingestible snacking calorie calculating unit 16 obtains α and β corresponding to user-specified desired snacking time and date according to the function data stored in the function data storage unit 23.

More specifically, the ingestible snacking calorie calculating unit 16 calculates the total value of calories obtained by weighting the calorie consumption by the user during the first period before the desired snacking time by the coefficient α which varies with time within the day and the expected calorie consumption by the user during the second period after the desired snacking time, and the calorie consumption (estimated daily calorie consumption) is obtained by weighting the total value by the coefficient β which varies with the time within the day. The total value (estimated calorie intake) of calorie intake by the user during the first period and expected calorie intake by the user during the second period are obtained. Then, the ingestible snacking calories are calculated on the basis of the difference between the obtained estimated calorie consumption and the obtained estimated calorie intake.

When the ingestible snacking calorie calculating unit 16 calculates the ingestible snacking calories corresponding to the desired snacking time on the basis of the conversion formula, the ingestible snacking calorie calculating unit 16 passes the value of the calculated ingestible snacking calories to the recommended food item selecting unit 17 in step S175.

(5) Selection of Food Item to be Recommended for Snacking

Under the control of the recommended food item selecting unit 17, the control unit 1 selects a food item in step S18 as follows. FIG. 9 is a flowchart for illustrating the processing procedure and processing content.

More specifically, the recommended food item selecting unit 17 first reads in step S181 information representing the history of food item selection in the past from the recommendation information storage unit 25, and recognizes a cluster selected for the previous snacking on the basis of the selection history information. Then, in step S182, a cluster other than the cluster selected for the previous snacking is selected.

Then, in step S183, the recommended food item selecting unit 17 refers to food item information stored in the food item information storage unit 24 and selects, from the food items belonging to the selected cluster, a food item having calories not more than the ingestible snacking calorie value calculated by the ingestible snacking calorie calculating unit 16.

For example, assume that the information shown in FIG. 3 is stored in the food item information storage unit 24 as food item information, and cluster ID=3 has been selected as a cluster at the time of the previous snacking, for example, cluster ID=2 will be selected this time. Then, a food item having calories not more than the ingestible snacking calories is selected from the group of food items belonging to the cluster ID=2. When for example the ingestible snaking calorie value is 80 [kcal], then the “baked pudding” is selected.

When the ingestible calorie value for snacking is set to the minimum value, and the minimum value is 40 [kcal], then the “potato” is selected among the food items belonging to the cluster with cluster ID=2. When the ingestible snacking calorie value is set to the minimum value, then a food item having at most minimum value may be selected among all clusters other than the cluster selected for the previous snacking. In this case, in the example shown in FIG. 3, the “candy” with the minimum calories is selected among the clusters with cluster ID=1.

Note that when no food item with a calorie level equal to or lower than the ingestible snacking calories can be found among the clusters other than the previously selected cluster, the previously selected cluster may be selected, and a food item other than the previously selected food item and having calories equal to or lower than the ingestible snacking calories can be selected from the cluster. When there are multiple food items which satisfy the conditions of clusters and ingestible snaking calories, these food items may be selected as recommended food item candidates.

The recommended food item selecting unit 17 stores the “food item name”, “calories”, and “image ID” of the food item selected as recommended food item information in the recommendation information storage unit 25. At the same time, the recommended food item selecting unit 17 updates the history of the recommended food item selection, or information indicating which food item belonging to which cluster was selected and when.

(6) Presentation of Recommended Food Items

When a food item satisfying the conditions is selected, the control unit 1, under the control of the recommended food item information output unit 18, reads the recommended food item information from the recommendation information storage unit 25, generates display data on the basis of the recommended food item information, and supplies the data from the input/output I/F 5 to the display unit 8B for display. In this case, the recommended food item information output unit 18 uses the “image ID” as a key to read the image of the corresponding food item from a food item image storage unit in the storage unit 2 and has the image of the food item included in display data and displayed.

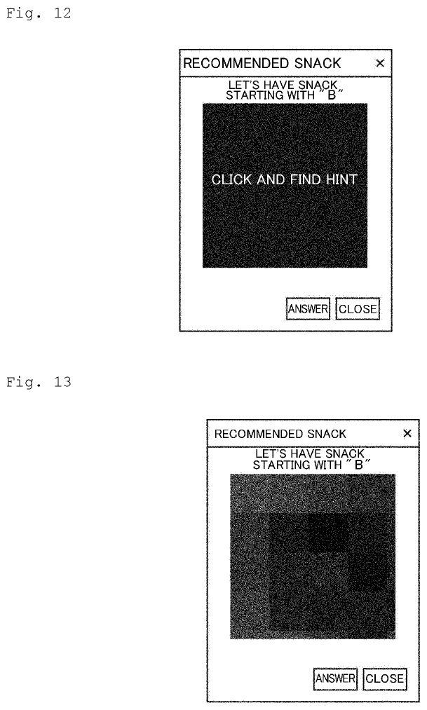

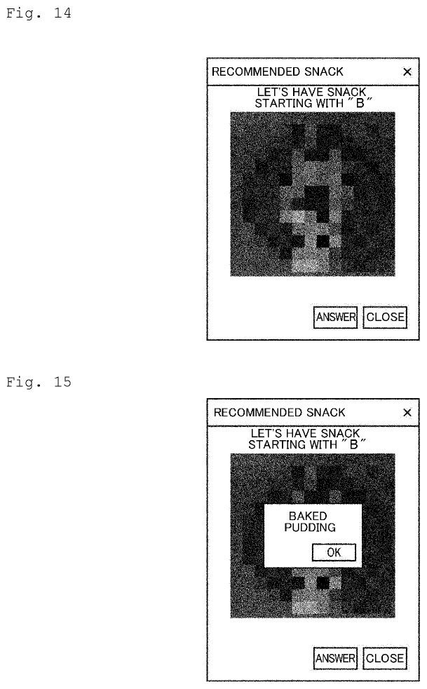

Note that various methods may be applied to display the recommended food item information other than the above method for displaying the food item name, the calories, and the image of the food item. For example, as shown in FIG. 12, only the initial letter of the food item is displayed without displaying the image of the food item. In this state, when the user clicks on the image display area, the food image is displayed mosaicked with a coarse grain size as shown in FIG. 13, and when the mosaic image is further clicked in the state, the food image is displayed mosaicked with a fine grain size as shown in FIG. 14. When the user clicks on the “Answer” button to input the name of the food item from the input unit 8A, and the input name of the food item is correct, the name of the correctly answered food item is displayed in the image display area as shown in FIG. 15.

According to the display method, the user can enjoy checking food items for snacking, the user is less likely to feel tired of food items despite their calorie restriction. The user may also easily remember food items and calories by associating them with images.

(Functions and Effects)

As in the foregoing, according to the embodiment, the food item information storage unit 24 stores food item information including multiple food items suitable for snacking which are classified into multiple clusters on the basis of similarity in the feature quantities for the shape, taste, and texture. The calories less than the difference between the sum of the calorie consumption by the user during the first period before the user-specified desired snacking time and the expected calorie consumption during the second period thereafter and the sum of the calorie intake by the user during the first period and the expected calorie intake during the second period are obtained as the ingestible snacking calories, and a food item which belongs to a cluster other than the cluster selected for the previous snacking and has calories less than the ingestible snacking calories is selected from the food item information and presented to the user.

Therefore, when selecting a food item suitable for intake at user-specified desired snacking time, a food item which belongs to a cluster different from the previously selected cluster and having calories less than the calculated ingestible snacking calories is selected. As a result, a food item of which the user is less likely to be tired can be selected for each snacking occasion, which can effectively support the user's health care efforts.

When calculating the ingestible snacking calories, the expected calorie consumption during the second period and the sum of the expected calorie consumption and the calorie consumption by the user during the first period are weighted by coefficients β and α, respectively which vary with the elapse of time within the day. In this way, the calorie consumption can be corrected according to the time of day within the day, which in turn makes it possible to accurately calculate the ingestible snacking calories in consideration of the diurnal characteristic of human calorie consumption.

[Modifications]

The present invention can have various modifications as follows.

(1) In the above description of the embodiment, the health care support function according to the present invention is provided in a mobile information terminal UT. For example, the health care support function may be provided on a sever device, and the server device may be configured to obtain exercise data and meal information from the user terminal via a network, select a food item suitable for snacking, and transmit information on the selected food item from the server device to the user terminal via the network for display.

(2) According to the embodiment, a group of food items suitable for snacking are classified into multiple clusters according to similarity in the feature quantities for shape, taste, and texture, but the feature quantities of each food item may be calculated in consideration of their nutrient contents, and the food item group may be classified into multiple clusters according to similarity in the feature quantities. In addition, the feature quantities may be weighted according to the user's preferences, gender, age group, region of residence, and other attributes.

(3) In the above description of the embodiment, a food item suitable for snacking is selected by way of illustration, while the invention is also applicable to the case of selecting a dessert for any of the three meals or selecting a food item suitable for a late-night snack.

(4) Alternatively, the configuration of the health care support device, the series of processing procedures and contents from obtaining of user's exercise data and meal information to selection and output of a recommended food item, the data arrangement of food item information, or the manner of presenting selected recommended food items may be modified in various manners without departing from the gist and scope of the present invention.

In short, the present invention is not limited by the above-described embodiment and can be modified in various manners without departing from the gist and scope thereof when implemented. The features of the embodiment may be combined as appropriate in every possible manner and carried out, in which case the effects may be combined. The embodiment includes inventions in various phases, and various inventions can be extracted by appropriately combining a plurality of elements disclosed herein.

### REFERENCE SIGNS LIST

UT Mobile information terminal
1 Control unit
2 Storage unit
3 Communication (I/F)
4 Sensor I/F
5 Input/output I/F
6 Sensor
7 Camera
8A Input unit
8B Display unit
9 Bus
11 Exercise data obtaining unit
12 Calorie consumption calculating unit
13 Meal information obtaining Unit
14 Calorie intake calculating unit
15 Expected calorie calculating unit
16 Ingestible snacking calorie calculating unit
17 Recommended food item selecting unit
18 Recommended food item information output unit
21 Exercise data storage unit
22 Meal information storage unit
23 Function data storage unit
24 Food item information storage unit
25 Recommendation information storage unit

1. A health care support device comprising a memory and a hardware processor,
the memory comprising a storage unit which stores information representing a plurality of food items in association with calories contained therein, the plurality of food items being classified into a plurality of clusters according to similarity in a feature quantity thereof,
the hardware processor being configured to carry out:
calculating ingestible calories which can be ingested at user-specified desired intake time according to the difference between estimated calorie consumption and estimated calorie intake by the user during a previously set period;
selecting, from the storage unit, a food item having contained calories less than the calculated ingestible calories and belonging to a cluster other than a cluster from which a food item has been selected at time before the desired intake time; and
outputting the information representing the selected food item.

2. The health care support device according to claim 1, wherein
the storage unit stores information representing the plurality of food items in association with the calories contained therein, the plurality of food items being classified into a plurality of clusters according to similarity in a feature quantity for at least one of the shape, taste, and texture.

3. The health care support device according to claim 1, wherein
in the step of calculating the ingestible calories, a total value of calorie consumption by the user during a first period before the desired intake time and expected calorie consumption by the user during a second period after the desired intake time is obtained as the estimated calorie consumption, while a total value of calorie intake by the user during the first period and expected calorie intake by the user during the second period is obtained as the estimated calorie intake, and the ingestible calories are calculated according to the difference between the estimated calorie consumption and the estimated calorie intake.

4. The health care support device according to claim 3, wherein
in the step of calculating the ingestible calories, the ingestible calories are calculated according to the difference between the calorie consumption weighted by a coefficient which varies with the elapse of time and the expected calorie intake.

5. The health care support device according to claim 1, wherein
the storage unit stores information representing a plurality of food items available for snacking in association with calories contained therein, the plurality of food items being classified into a plurality of clusters according to similarity in a feature quantity thereof,
in the step of calculating the ingestible calories, it is determined whether the desired intake time for snacking is included within a prescribed time period before and after a regular meal, the ingestible calories are set to a previously set minimum value when it is determined that the time is included, and the ingestible calories are calculated according to the difference between estimated calorie consumption and the estimated calorie intake by the user when it is determined that the time is not included.

6. A health care support method carried out by a health care support device comprising a memory and a hardware processor, the method comprising the steps, carried out by the hardware processor, of:
storing, in the memory, information representing a plurality of food items in association with calories contained therein, the plurality of food items being classified into a plurality of clusters according to similarity in a feature quantity thereof;
calculating ingestible calories which can be ingested at user-specified desired intake time according to the difference between estimated calorie consumption and estimated calorie intake by the user during a previously set period;
selecting, from the memory, a food item having contained calories less than the calculated ingestible calories and belonging to a cluster other than a cluster from which a food item has been selected at time before the desired intake time; and
outputting the information on the selected food item.

7. A program for use in a health care support device comprising a memory and a hardware processor, the program causing the hardware processor to carry out:
storing, in the memory, information representing a plurality of food items in association with calories contained therein, the plurality of food items being classified into a plurality of clusters according to similarity in a feature quantity thereof;
calculating ingestible calories which can be ingested at the desired intake time according to the difference between estimated calorie consumption and estimated calorie intake by the user during a previously set period;
selecting, from the memory, a food item having contained calories less than the calculated ingestible calories and belonging to a cluster other than a cluster from which a food item has been selected at time before the user-specified desired intake time; and
outputting the information representing the selected food item.
